(19) United States
(12) Patent Application Publication
(10) Pub. No.: US 2022/0218630 A1
(43) Pub. Date: Jul. 14, 2022

(54) METHODS AND FORMULATIONS FOR TREATING VISION DISORDERS

(71) Applicant: JeniVision Inc., Irvine, CA (US)

(72) Inventors: David F. Woodward, London (GB); Weizhen Wang, Irvine, CA (US)

(21) Appl. No.: 17/610,368

(22) PCT Filed: Jun. 9, 2020
(86) PCT No.: PCT/US2020/036775

§ 371 Date: Nov. 10, 2021

Related U.S. Application Data
(60) Provisional application No. 62/859,682, filed on Jun. 10, 2019.

(51) Int. Cl. A61K 31/137; A61K 31/165; A61K 31/439; A61K 31/46; A61K 31/4704; A61K 45/06; A61K 9/00; A61P 27/10

(52) U.S. Cl. CPC A61K 31/137; A61K 31/165; A61K 31/439; A61P 27/10; A61K 31/4704; A61K 45/06; A61K 9/0051; A61K 31/46

(57) ABSTRACT

Ocular formulations comprising long-acting β-adrenoceptor agonist (LABA) compounds, long-acting muscarinic antagonists (LAMA), and combinations thereof are provided. Methods for treating vision disorders, with LABAs, LAMAs, and combinations thereof are also provided.

### BACKGROUND

It is important to take into account that myopia, amblyopia, and related vision disorders emerge in childhood. It is at this early stage of life that an effective means of correction should be introduced. Currently, a common treatment is wearing spectacles but this is only a temporary measure and eyesight can continue to worsen, requiring spectacles of ever increasing strength. Physical changes to the eyeball can occur that can result in serious eye conditions. Therefore, more permanent forms of correction of such conditions have been sought, such as the use of the anti-cholinergic antagonist, atropine, to treat myopia, amblyopia, and related conditions at an early age and especially in Asia. However, atropine can lose effect with chronic usage and has a number of unwanted side effects in the eye and systemically, cardiac side effects being potentially life-threatening. In short, atropine can have undesirable side effects that should be avoided in pediatric ophthalmology and the current approach largely relies on reduced dosing.

### SUMMARY

There is a pressing need for safe, effective, and more convenient alternatives for treating many vision disorders. Long-acting β-adrenoceptor agonists (LABAs) can provide a safe and highly effective replacement for current treatments for a wide range of vision disorders. Long-acting muscarinic antagonists (LAMAs) are improved atropine-like compounds and their clinical performance may be improved by addition of a LABA or other β-adrenoceptor agonist.

Methods of drug application and formulations for treating vision disorders by employing LABAs, LAMAs, and a combinations of both drug classes are provided herein. These methods relate to the treatment of vision disorders like pseudo-myopia, pre-myopia, cycloplegia, and myopia. In addition, other disorders, such as anisometropia and amblyopia, characterized by abnormal ciliary muscle tone can be treated using the compounds described herein. In addition to improving visual acuity, the methods described herein can attenuate the axial elongation and abnormal development of the eyeball that is associated with vision disorders. Such remedial benefits are achieved by the administration of a LABA, a LAMA or a combination thereof. In cases of myopia, a LABA maintains stable and titratable relaxation of the ciliary muscle preventing conversion of myopia to presbyopia. By employing the compounds described herein, light can be more accurately focused on the retina and clearer vision can be achieved. Eye strain, which is a major exacerbating factor, can also be relieved.

In the case of anisometropia, amblyopia, and certain related vision disorders, the pathophysiology is one eye is continually underused. This underuse is progressive. The underused eye is commonly referred to as a “lazy” eye. The other eye may be referred to as the “normal,” “unaffected,” “strong,” “preferred,” or “dominant” eye. Eventually anisometropia, amblyopia, and related vision disorders result in the patient entirely relying on the “strong” eye, the “lazy” eye falling out of use. The therapeutic objective is to prevent the “lazy” eye from falling out of use and prevent the patient from developing monocular vision. This is currently achieved by application of a patch or atropine to the “strong” or “dominant” eye, which results in reduced use and reduced over-reliance on this “strong” or “preferred” eye, increasing participation of the “weaker” eye to restore binocular vision.

As provided in the methods described herein, a LABA can similarly be used to compromise vision in the “normal” or “strong” eye, described herein by the terminology “β-adrenoceptor agonist penalization.”

Methods of treating compromised vision by modulating ciliary muscle tone using a LABA are also described. Such compromised vision conditions can include, as non-limiting examples, anisometropia, amblyopia, pseudomyopia, and myopia. LABA compounds are those described as LABAs according to the American Academy of Allergy, Asthma, and Immunology (AAAAI Allergy and Asthma Medication Guide, 2016) and include formoterol, salmeterol, arformeterol, and olodaterol. The amount of LABA given to subjects can range from about 0.001% to about 10% (weight/volume, e.g., mg/mL). Additional non-limiting delivery methods that can provide drug delivery to the ciliary muscle include, e.g., anterior chamber implants, subconjunctival injection, subconjunctival/suprachoroidal implants, and topical application to the periorbital region (see, e.g., U.S. Pat. No. 9,820,954).

Without being bound by theory, it is believed that a LABA, as described herein, can reduce the resting tone of the ciliary muscle, thereby reducing the ciliary muscle contractile and spasmodic episodes associated with pseudomyopia. To the contrary, short acting β-adrenoceptor agonists, such as salbutamol (Rekik, WO2018/007864 A1, 2018) and isoproterenol are likely to produce relatively transient effects and a predisposition to breakthrough ciliary muscle spasm. The opposing effect of the LABA, as described herein, on ciliary muscle contraction can provide effective treatment for myopia, thus, reducing or eliminating the need for using spectacles or contact lenses, or slowing the rate of increased prescription strength of corrective lenses. The risk of retinal detachment, myopia induced retinopathies, and glaucoma can be mitigated or prevented by employing the methods provided herein. Repeated episodes of retinal detachment can lead to blindness. However, the increase in axial length of the globe that can occur in myopia and amblyopia can be attenuated or eliminated following treatment with a LABA using the methods described herein.

Anti-muscarinic drugs, notably atropine, have been employed to treat myopia and amblyopia but, although effective, can cause side effects, such as mydriasis, dry eye, and photophobia. The incidence of mydriasis may be reduced by using a LABA, optionally in combination with anti-muscarinic drugs. Anti-muscarinic drugs can also reduce lacrimal gland secretion and thereby cause “dry eye.” Indeed, atropine has been used to create an animal model of dry eye (Burgalassi et al. Development of a simple dry eye model in the albino rabbit and evaluation of some tear substitutes. Ophthalmol Res. 31: 229-235, 1999). By using a long-acting β-adrenoceptor therapy in place of, or in addition to, anti-muscarinic agents dry eye side effects can be avoided reduced or eliminated. Moreover, anti-muscarinic agents can produce serious cardiac side effects that can be more apparent in small children. Of particular concern is an increase in uncontrolled heart rate, which can be fatal in some instances. LABAs are known to be cardiac safe in children and are already widely used in the treatment of juvenile asthma. Thus, as described herein, a LABA can be used in combination with an anti-muscarinic agent to reduce the dose required and thereby reduce the unwanted side-effects associated with anti-muscarinic drugs. Formulations are provided having concentrations of a LABA and/or a LAMA from about 0.0001% to about 10% (weight/volume). Methods and formulations described herein can include combination of one or more LABAs (or one or more LABAs and one or more anti-muscarinic agents) and, optionally, with α-adrenergic compounds, such as, e.g., brimonodine.

As described herein, vision disorders, as generally described, may be effectively treated by the use of LABAs, LAMAs, or combinations thereof. Treatment may be binocular or monocular according to the vision disorder that may be remedied by altering, for example, ciliary muscle tone. A number of drug delivery methods can be used including, but not limited to, eye-drops and implants.

Provided is a method for treating a vision disorder in a subject in need of vision correction. The method includes administering a LABA, a LAMA, or a combination thereof to one or both eyes of the subject.

The method may optionally include one or more of the following features. The administration can be ocular or periocular. The vision disorder can be a disorder that can be treated by ciliary muscle modulation in an affected or non-affected eye of the subject. The disorder can be selected from the group consisting of myopia, premyopoia, pseudomyopia, cycloplegia, antimetropia, exophoria, amblyopia, anisometropia, esoptropia, exoptropia, Duane's syndrome I, Duane's syndrome II, Brown's syndrome, ocular complications from surgery, injury to the eye or fracture of the orbital bone, a vision disorder resulting from retinal detachment, a vision disorder resulting from cataract, a vision disorder associated with diabetes, a vision disorder associated with myasthenia gravis, and a vision disorder associated with Grave's disease. The LABA, LAMA, or a combination thereof can be administered to a subject's affected eye, unaffected eye, or both. The disorder can be selected from the group consisting of anisometropia, amblyopia, esoptropia, exoptropia, and co-morbities thereof. The disorder can be selected from the group consisting of myopia, pseudomyopia, antimetropia, and exophoria. The LABA, LAMA, or a combination thereof can be administered to the eye as a topical ocular formulation. The LABA, LAMA, or a combination thereof can be administered to the eye as an eye drop. The LABA, LAMA, or a combination thereof can be administered to the eye by topical application to the periorbital skin. The LABA, LAMA, or a combination thereof can be administered to the eye as an implant. The implant can be an ocular interior chamber implant or a suprachoroidal implant. The LABA can be selected from the group consisting of albuterol, formoterol, salmeterol, arformeterol, olodaterol, and combinations thereof. The LAMA can be selected from the group consisting of tioptropium, aclidinium, glycopyrrolate an combinations thereof. Combinations of an LABA and LAMA can be in the form of single hybrid molecules. Single hybrid molecules exhibiting both muscarinic antagonist and β-adrenergic properties (MABAs) may be selected from batefenterol, AZD2115, and AZD8871.

The method can optionally further include, in addition to administration of a LABA and/or a LAMA, administering a muscarinic antagonist to an eye of the subject. The method can optionally include one or more of the following features. The muscarinic antagonist can be administered to a same eye of the subject to which the LABA is administered. The muscarinic antagonist can be selected from the group consisting of atropine, scopolamine, hydroxyzine, ipratropium, tropicamide, pirenzepine, diphenhydramine, doxylamine, dimenhydrinate, dicyclomine, flavoxate, oxybutynin, tiotropium, cyclopentolate, atropine methonitrate, trihexyphenidyl, tolterodine, solifenacin, darifenacin, benztropine, mebeverine, procyclidine, aclidinium bromide, and combinations thereof. The muscarinic antagonist can be atropine. Optionally, the subject can be a subject that was previously treated with atropine. Optionally, a dose of atropine used in combination with the LABA can be reduced relative to the dose of atropine administered to the subject prior to treatment with the LABA.

The method can optionally further include, in addition to administration of the LABA and/or LAMA, administering an α-adrenergic agonist to the subject. The method can optionally include one or more of the following features. The α-adrenergic agonist can be selected from the group consisting of methoxamine, midodrine, oxymetazoline, metaraminol, phenylephrine, clonidine, guanfacine, guanabenz, guanoxabenz, guanethidine, xylazine, mizanidine, medetomidine, methyldopa, methylnorepinephrine, fadolmidine, dexmedetomidine, amidephrine, amitraz, anisodamine, apraclonidine, brimonidine, cirazoline, detomidine, epinephrine, ergotamine, etilefrine, indanidine, lofexidine, medetomidine, mephentermine, metaraminol, methoxamine, mivazerol, naphazoline, norepinephrine, norfenefrine, octopamine, oxymetazoline, phenylpropanolamine, propylhexedrine, rilmenidine, romifidine, synephrine, talipexole, salts thereof, and combinations thereof. The α-adrenergic agonist can be brimonodine.

The method can optionally further include, in addition to administration of the LABA, LAMA and/or α-adrenergic agonist, administering a phosphodiesterase (PDE) inhibitor to the subject. The method can optionally include one or more of the following features. The phosphodiesterase (PDE) inhibitor can be selected from the group consisting of vinpocetine, erythro-9-(2-hydroxy-3-nonyl)adenine (EHNA), 2-[(3,4-dimethoxyphenyl)methyl]-7-[(2R,3R)-2-hydroxy-6-phenylhexan-3-yl]-5-methyl-1H-imidazo[5,1-f][1,2,4]triazin-4-one, oxindole, 9-(6-Phenyl-2-oxohex-3-yl)-2-(3,4-dimethoxybenzyl)-purin-6-one (PDP), inamrinone, milrinone, enoximone, anagrelide, cilostazol, and pimobendan, mesembrenone, rolipram, ibudilast, piclamilast, luteolin, drotaverine, roflumilast, apremilast, crisaborole, sildenafil, tadalafil, vardenafil, udenafil, avanafil, dipyridamole, quinazoline, papaverine, and combinations thereof. The phosphodiesterase inhibitor can be theophylline.

Also provided are ocular formulations comprising a LABA. The β-adrenoceptor agonist can be selected from the group consisting of olodaterol, albuterol, formoterol, salmeterol, bambuterol, clenbuterol, protokylol and ultra-long-acting arformeterol, carmoterol, indacaterol, and combinations thereof. The LABA can be present at a concentration of from about 0.0001% to about 10% w/v. The formulation can be a topical ocular formulation. The topical ocular formulation can be in the form of an eye drop. The topical ocular formulation can be in the form of a periorbital formulation.

The ocular formulation can optionally further include a muscarinic antagonist. The muscarinic antagonist can be selected from the group consisting of atropine, scopolamine, hydroxyzine, ipratropium, tropicamide, pirenzepine, diphenhydramine, doxylamine, dimenhydrinate, dicyclomine, flavoxate, oxybutynin, tiotropium, cyclopentolate, atropine methonitrate, trihexyphenidyl, tolterodine, solifenacin, darifenacin, benztropine, mebeverine, procyclidine, aclidinium bromide, and combinations thereof. The muscarinic antagonist can be atropine. The muscarinic antagonist can be present at a concentration of from about 0.0001% to about 10% (w/v). Optionally, the ocular formulation can include a muscarinic antagonist/02-agonist hybrid molecule, which may be selected from batefenterol, AZD2115, and AZD8871.

Also provided is a method for improving visual acuity in an affected eye of a subject having myopia, comprising administering to the subject a LABA, a LAMA, or a combination thereof or administering a pi-adrenergic agonist/muscarinic antagonist in a single hybrid molecule to the subject.

The methods described herein provide several advantages. First, the methods can provide safe and effective permanent (or, in some cases, long-term, semi-permanent) treatment for the vision disorders by modulating ciliary muscle tone. Thus, unlike corrective lenses, the methods described herein can prevent the development of more serious eye conditions, such as retinal detachment, myopic retinopathies (such as neovascularization, lattice degeneration and rips/tears), staphyloma, cataracts, and glaucoma, which may develop due to progressive physical changes to the eyeball that may occur with the vision disorders described herein if left untreated or if treated symptomatically by other means like corrective lenses.

Second, the methods described herein can reduce the reliance on corrective lenses of a subject having such vision disorders. For example the methods described herein can lead to improved vision, including improved visual acuity. Abnormal axial elongation of the eyeball may be avoided and thereby co-morbidities, notably serious, sight-threatening retinal conditions, may also be avoided.

Third, the methods described herein can reduce or even eliminate the use of, or reliance on, treatments that can have more serious side effects. For example, the commonly-used muscarinic agent, atropine, can produce ocular side effects such as dry eye, mydriasis, photophobia. When absorbed into the blood stream, atropine may cause cardiac arrest as a result of unregulated sympathetic neurostimulation of heart rate; this can result from reduction or prevention of parasympathetic neuronal input, which normally negatively regulates heart rate. Atropine is a derivative of Atropo belladonna (Deadly Nightshade), one of the most toxic plants known (e.g., belladonna poisoning). Its unrestricted use in young children could be fatal. The methods described herein can provide safe, effective alternative treatment for the vision disorders, with reduced dosages of atropine or other muscarinic antagonists. As described herein, the methods can provide safe, effective, optimized treatment for vision disorders without the use of atropine.

Fourth, the methods described herein can provide a sustained effect with a long duration of action in the subject's eye, allowing for increased safety. The methods described herein can provide long-duration ciliary muscle modulation using a LABA, a LAMA, or a combination thereof administered together or separately or administered as a hybrid molecule with β-adrenergic agonist and muscarinic antagonist properties. The methods and formulations described herein can optimally reduce and re-set the tone of the ciliary muscle, thereby reducing ciliary muscle contractile and spasmodic episodes. The methods and formulations described herein produce greater than transient effects on ciliary muscle spasm. The methods and formulations described herein do not result in breakthrough ciliary muscle spasm nor convert the myopic patient to presbyopic.

Fifth, the methods described herein can provide safer and practical treatment options for pediatric patients by providing a long or extended duration of activity in the eye.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1A is a graph showing effect of salmeterol on a paired monkey longitudinal ciliary muscle preparation contracted by carbachol FIG. 1B is a graph showing effect of tiotropium on a paired monkey longitudinal ciliary muscle preparation contracted by carbachol.

FIG. 1C is a graph showing effect of salmeterol and tiotropium combined on a paired longitudinal ciliary muscle preparation contracted by carbachol.

### DETAILED DESCRIPTION

Provided herein are methods and formulations for treating vision disorders by employing long-acting β-adrenoceptor agonist (LABA) compounds, long-acting muscarinic antagonists (LAMA), and combinations thereof.

The present disclosure is directed to the treatment of vision disorders that can be modified (e.g., the symptoms or course of the disease can be modified) by ciliary muscle modulation, including modulation and regulation of ciliary muscle tone, such as, for example, in pseudomyopia, myopia, and other disorders, such as anisometropia and amblyopia. The provided methods include administration of LABAs, LAMAs, and combinations thereof administered separately or together or as a hybrid molecule with β-adrenergic agonist and muscarinic antagonist properties, to a subject having the vision disorder.

A critically important tissue involved in myopia is the ciliary muscle. The function of the ciliary muscle is to control the spherical shape of the lens, which controls the focal point of light passing through the eye. The ability of the eye to provide focus on objects at varying distances from the eye is termed accommodation, a key ocular function.

Myopia often begins as pseudomyopia. Pseudomyopia describes temporary shifts in light refraction due to transient spasm of the ciliary muscle and an inability of the ciliary muscle to properly relax. Pseudomyopia may occur as a result of excessive parasympathetic neuronal activity or as a result of eye strain or fatigue. This continued strain often gradually results in an elongated globe. The constant strain of close-up work in children is an exacerbating factor that extends the axial length of the globe.

Corrective lenses, such as spectacles and contact lenses, are frequently used to treat myopia but these typically only shift the focal point such that light converges on the retina rather than in the vitreous humor. In short, spectacles and contact lenses typically only improve vision by changing the point of focus rather than improving ciliary muscle control of accommodation. The eye then tends to adjust to the creation of a new focal point and gradually this results in elongation of the globe. As a result, prescription lenses typically become stronger, ciliary muscle function is further compromised, and a negative cycle can ensue. The ciliary muscle can remain stressed with less ability to relax. Unwanted side effects may eventually occur including retinal detachment, myopic retinopathies, and glaucoma.

Current medical therapies to relieve myopia involve reducing parasympathetic neuronal tone to relax the ciliary muscle. This is typically achieved by using muscarinic receptor antagonists, almost invariably atropine. Atropine can cause dry eye, mydriasis, and photophobia and, when absorbed into the blood stream, may produce serious cardiac side effects. Effects on heart rate can, in certain cases, be fatal. Atropine may also become less effective with the passage of time. These characteristics make atropine particularly undesirable, and even potentially dangerous, in pediatric patients, the population in which corrective treatment should be used.

While parasympathetic nerves are the primary controlling influence on ciliary muscle tone, sympathetic nerves provide opposing regulatory input. The sympathetic neuronal input relaxes the ciliary muscle, which opposes the contractile activity of parasympathetic nerves. One of the several target receptors that mediate ciliary muscle relaxation is the β-adrenoceptor (Zetterström and Hahnenberger, Exp Eye Res 46, 421-430, 1988). LABAs can provide an approach to providing stable control of ciliary muscle tone that can be used in combination with, or instead of, atropine treatment.

Amblyopia, commonly known as lazy eye also manifests in childhood and, if left untreated, will result in monocular vision in adults. Anisometropia is often a precursor for amblyopia. In amblyopia and related vision disorders, one eye is used in preference to the contralateral (or lazy) eye. A “lazy” eye has weaker vision and falls into disuse. The development of a “lazy” eye has many possible origins. These can include poor vision due to poor accommodation, suboptimal eye movement due to extraocular muscle malfunction, or a visual cortex issue. Regardless of the pathophysiological origin of the “lazy” eye, the treatment is the same.

One common treatment includes using separate prescriptions for corrective lenses for the individual eyes. Spectacles are not a long term solution and meaningfully improve vision only in the dominant eye. The subject typically remains amblyopic.

Another common strategy that brings positive results includes weakening the “stronger” dominant eye or non-affected eye, so that the lazy eye or affected eye, is forced to function. One approach to weakening the dominant eye is to place a patch over the dominant eye, such as for 6 hour periods. However, patch-wearing compliance in pediatric patients can be challenging. An additional approach is termed atropine penalization, which involves compromising the focus of the “stronger” dominant eye by altering ciliary muscle tone and thereby accommodation. LABAs, LAMAs, and combinations thereof or a hybrid molecule with β-adrenergic agonist and muscarinic antagonist properties can be useful solutions for providing more stable “penalization.”

As described in the methods herein, LABA, LAMA, and a combination thereof administered together or separately or as a hybrid molecule with β-adrenergic agonist and muscarinic antagonist properties can modulate ciliary muscle tone to allow for treatment of applicable vision disorders. In the methods described herein, treatment with LABA, LAMA, and a combinations thereof or a hybrid molecule with β-adrenergic agonist and muscarinic antagonist properties can improve visual acuity, attenuate elongated axial growth and restrict the abnormal development of the eye that is associated with the vision disorder. In treating a subject having myopia, a LABA, a LAMA, or combinations thereof or a hybrid molecule with β-adrenergic agonist and muscarinic antagonist properties can maintain stable relaxation of the ciliary muscle and thereby improve accommodation such that light is accurately focused on the retina and clearer vision is achieved. In addition, long-lasting effects provided by LABAs, LAMAs, and combinations thereof may result in more rapid restoration of use in the “lazy” eye and promote binocular vision. In treating a subject having anisometropia or amblyopia, application to the dominant eye can result in reduced over-reliance on the “stronger” dominant preferred eye and increase participation of the “weaker” eye to restore binocular vision. Eye strain, which is a major exacerbating factor, can also be relieved.

A range of different strengths (doses) of a LABA, a LAMA, or both when present in a combination are contemplated. The doses of LABAs, LAMAs, combinations thereof, or a hybrid molecule with β-adrenergic agonist and muscarinic antagonist properties may be based on the individual needs of individual subjects. In a single subject, vision in individual eyes may differ and thus dosage needs may also differ. As used herein, a dose refers to the amount of active ingredient given to an individual at each administration. The dose will vary depending on a number of factors, including the range of normal doses for a given therapy, frequency of administration; size and tolerance of the individual; severity of the condition being treated; risk of side effects; and the route of administration. One of skill will recognize that the dose can be modified depending on the above factors or based on therapeutic progress.

Provided herein are methods for treating a vision disorder in a subject. The methods for treating a vision disorder in a subject include administering a LABA, a LAMA, a combination thereof, or a single drug that exhibits both activities to the subject. The drugs can be formulation in a suitable composition for administration to the subject, e.g., the eye of the subject. The composition can include one or both drugs. If administered separately, the drugs are formulated in separate compositions. Methods are also provided for treating compromised vision by modulating ciliary muscle tone. For example, methods are provided for treating compromised vision in a subject by modulating ciliary muscle tone using a LABA, LAMA, a combination thereof, or a single drug that exhibits both activities.

The methods described herein can be useful for treating vision disorders that can be modified by ciliary muscle modulation in an affected or a non-affected eye. Examples of vision disorders where monocular administration to the non-affected eye therapy is preferable include anisometropia, amblyopia, antimetropia, esoptropia, exoptropia, Duane's syndromes I/II, Brown's syndrome, and monocular eye trauma such as an orbital bone fracture. Binocular treatment may be preferable for treating pseudomyopia, myopia, exophoria, and vision complications resulting from systemic diseases such as Grave's disease, myasthenia gravis, and diabetes. Different drug “strengths” may be required according to differences apparent at visual examinations. A LABA, LAMA, a combination comprising a β-adrenoceptor agonist and a LAMA, or a single drug that exhibits both activities can be administered to an affected eye of a subject having a vision disorder. Without wishing to be bound by theory, it is believed that a LABA, LAMA, a combination comprising a β-adrenoceptor agonist and a LAMA, or a single drug that exhibits both activities can modulate ciliary muscle tone in an affected eye, thereby alleviating at least one symptom of the vision disorder. Administration of a LABA, LAMA, a combination comprising a β-adrenoceptor agonist and a LAMA, or a single drug that exhibits both activities to an affected eye of a subject with a vision disorder can improve the subject's vision. Optionally, a LABA. LAMA, a combination comprising a LABA, LAMA, or a single drug that exhibits both activities can be administered to a non-affected eye of a subject having a vision disorder. Without being bound by theory, it is believed a LABA, LAMA, a combination comprising a LABA and a LAMA, or a single drug that exhibits both activities can modulate ciliary muscle tone in a non-affected eye, thereby alleviating at least one symptom of the vision disorder. Administration of a LABA, LAMA, a combination comprising a LABA and a LAMA, or a single drug that exhibits both activities to a non-affected eye of a subject with a vision disorder can improve the subject's vision in an affected eye. As used herein, an affected eye is an eye of a subject considered to be an eye exhibiting one or more symptoms associated with a vision disorder. For example, non-limiting symptoms may include irregular eyeball shape (e.g., elongation), reduced visual acuity (e.g., visual acuity of less than 20/20, less than 20/25, less than 20/30, less than 20/40, less than 20/50, less than 20/70, less than 20/100, or less than 20/200). A non-affected eye is typically an eye that does not exhibit one or more symptoms, limited or aberrant motility, or reduced visual acuity associated with a vision disorder (e.g., the disorder in the subject's other eye). However, a non-affected eye may exhibit some collateral symptoms such as eye strain, due to the non-affected eye's role in vision compensation for the affected eye. A non-affected eye can be considered to be a dominant eye, such as in a subject having amblyopia (lazy eye).

LABAs suitable for use in the provided methods, formulations, and compositions may have a long, lipophilic side-chain and generally exhibit a duration of 6-15 hours in lung smooth muscle. Non-limiting examples of LABAs include albuterol, formoterol, salmeterol, olodatero. Further, non-limiting ultra-long-acting β-adrenoceptor agonists suitable for use in the methods, formulations, and compositions described herein include arformoterol, carmoterol, and indacaterol. Combinations of β-adrenoceptor agonists are also envisioned. Exemplary β-adrenoceptor agonist compounds include those described as such according to the American Academy of Allergy, Asthma, and Immunology (AAAAI Allergy and Asthma Medication Guide, 2016). A LABA can provide consistent, safe, and/or effective reduced resting tone of the ciliary muscle, thereby providing treatment, via administration to an affected eye or non-affected eye, of a subject having a vision disorder.

Previously, anti-muscarinic agents, almost invariably atropine, have been employed to treat myopia and amblyopia. More recently other muscarinic antagonists, including the long-acting muscarinic blocking agents umeclidinium and tiotropium have been contemplated for the treatment of myopia (WO Pat. No. 2018/17445; WO Pat. No. 2019/018749). Although effective, these agents can cause side effects such as mydriasis and photophobia. The incidence of mydriasis and/or photophobia can be reduced by administering a LABA in combination with, or instead of, an anti-muscarinic agent. Anti-muscarinic agents can also reduce lacrimal gland secretion and thereby cause dry eye. Conversely, dry eye symptoms can be minimal or even improved with administration of LABA therapy. Moreover, anti-muscarinic agents can produce serious cardiac side effects. Such side effects can be particularly pronounced and dangerous in small children, the population typically administered drug-based therapy for the vision disorders described herein. Administration of long-acting β-agonists in place of anti-muscarinic agents can avoid such serious cardiac side effects. Thus, a LABA can be administered in combination with an anti-muscarinic agents to reduce the dose of anti-muscarinic agents required and thereby reduce the unwanted side-effect risks associated with anti-muscarinic agents such as atropine.

LABAs are used in adequate doses to provide a prolonged residence time in the eye and/or modified to increase the duration of activity by increasing bioavailability. The duration of action of LABAs may be improved by formation of an ion pair complex, or utilizing an excipient that increases penetration into the globe and drug delivery to the ciliary muscle. A LABA can permit improved therapeutic efficiency, by virtue of a prolonged duration of action. At the prescribed dose, a consistently optimal effect on the ciliary muscle tone can be achieved, without rebound, spasm, or loss of activity due to short bio-availability of bio-activity of the drug in the ciliary muscle tissue. It has been established, for example, that the long-acting effects of salmeterol are based upon unique pharmacology (Coleman R. A. On the mechanism of the persistent action of salmeterol; what is the current position? Br J Pharmacol 2009; 158: 180-182), not its bioavailability.

A LABA, a LAMA, a combination thereof, or a single drug that exhibits both activities can be administered to an affected eye or to a non-affected eye of a subject as a topical ocular formulation. Optionally, the topical ocular formulation can be administered as an eye drop in an affected eye or non-affected eye of a subject.

The periorbital administration route (U.S. Pat. No. 9,820,954) of a LABA, LAMA, a combination thereof, or a single drug that exhibits both activities can be used.

The topical ocular formulation can be administered by topical application to the periorbital skin of an affected eye or non-affected eye of a subject. In periorbital application, the drug formulation is applied over the upper and lower periorbital skin of each eye and is not applied to the upper or lower eyelid or eyelid margin. In some embodiments, the formulation is applied over the upper and lower periorbital skin of each eye. Periorbital application of an ocular formulation is further described in U.S. Pat. No. 9,820,954.

Additional delivery methods can be used for the methods described herein to provide delivery of a LABA, a LAMA, a combination thereof, or a single drug that exhibits both activities. When given to an affected eye or non-affected eye of a subject include implants, e.g., anterior chamber implants, subconjunctival implants, suprachoroidal implants; and injection, e.g., subconjunctival injection.

Non-limiting exemplary s-adrenoceptor agonists that can be used in the methods described herein include LABAs such as arformoterol, bambuterol, protokylol, clenbuterol, formoterol, salmeterol, or ultra-long-acting β-adrenoceptor agonists such as abediterol, carmoterol, indacaterol, olodaterol, vilanterol, salts thereof, and combinations thereof. The LABA can be selected from the group consisting of formoterol, salmeterol, arformeterol, olodaterol, salts thereof, and combinations thereof. Additional β-adrenoceptor agonists include isoproterenol, denopamine, dobutamine, dopexamine, prenalterol, xamoterol, bupheninie, fenoterol, isoetarine, levalbuterol, metaproterenol, pirbuterol, procaterol, terbutaline, and ritodrine. Optionally, the LABA is salmeterol. Optionally, the LABA is formoterol. Optionally, the LABA is a β₂-adrenoceptor agonist. Optionally, the LABA is a β₂/β₃-adrenoceptor agonist. Optionally, the LABA is a β₂/β₁-adrenoceptor agonist.

Optionally, certain LABAs can be safely used in the pediatric ophthalmology. According to the American Academy of Allergy, Asthma, and Immunology (AAAAI Allergy and Asthma Medication Guide, 2016), salmeterol and formoterol are approved for use in children as young as 4 and 5, respectively.

The LABA can be present in a formulation in a concentration of ranging from about 0.001% to about 10% (weight/volume). For example, about 0.001% to about 9%, about 0.001% to about 8%, about 0.001% to about 7%, about 0.001% to about 6%, about 0.001% to about 5%, about 0.001% to about 4%, about 0.001% to about 3%, about 0.001% to about 2%, about 0.001% to about 1%, about 0.001% to about 0.5%, about 0.001% to about 0.1%, 0.01% to about 9%, about 0.01% to about 8%, about 0.01% to about 7%, about 0.01% to about 6%, about 0.01% to about 5%, about 0.01% to about 0.5%, about 0.01% to about 4%, about 0.01% to about 3%, about 0.01% to about 2%, about 0.01% to about 1%, about 0.01% to about 0.5%, about 0.01% to about 0.1%, about 0.01% to about 0.05%, about 0.05% to about 2%, about 0.05% to about 1%, about 0.05% to about 0.09%, about 0.01% to about 0.08%, about 0.01% to about 0.075%, about 1% to about 5%, about 2% to about 5%, about 3% to about 5% w/v, about 2% to about 8%, about 3% to about 7% w/w, about 4% to about 6%, about 5% to about 10%, about 5% to about 9%, or about 5% to about 8%, about 5% to about 7%, or about 5% to about 6% of the composition. In some embodiments, the LABA is present in an amount of about 0.01%, about 0.02%, about 0.03%, about 0.04%, about 0.05%, about 0.06%, about 0.07%, about 0.08%, about 0.09%, about 0.1%, about 0.11%, about 0.12%, about 0.13%, about 0.14%, about 0.15%, about 0.16%, about 0.17%, about 0.18%, about 0.19%, about 0.2%, about 0.3%, about 0.4%%, about 0.5%, about 0.6%, about 0.8%, about 0.9%, about 0.001%, or about 0.002%, about 0.003%, about 0.004%, about 0.005%, about 0.006%, about 0.007%, about 0.008%, about 0.009%, about 0.015%, about 0.025%, about 0.035%, about 0.045%, about 0.055%/s, about 0.065%, about 0.075%, about 0.085%, or about 0.095% w/v of the composition. Optionally, the LABA is present in an amount of about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% w/v of the composition; where w/v is mg/mL. Further subdivision within these ranges is also contemplated; for example, the LABA is present in an amount of from about 0.001% to about 0.1% (w/v), or about 0.0015%, 0.002%, 0.0025%, 0.003%, 0.0035%, 0.004%, 0.0045%, 0.005%, 0.0055%, 0.006%, 0.0065%, 0.007%, 0.0075%, 0.008%, 0.0085%, 0.009%, 0.0095%, 0.01%, 0.015%, 0.02%, 0.025%, 0.03%, 0.035%, 0.04%, 0.045%, 0.05%, 0.055%, 0.06%, 0.065%, 0.07%, 0.075%, 0.08%, 0.085%, 0.09%, 0.095%, or 0.1%, etc. Smaller incremental subdivisions are also contemplated; for example, the LABA is present in an amount of from about 0.2% to about −0.3% (w/v), or about 0.2%, 0.21%, 0.22%, 0.23%, 0.24%, 0.25%, 0.26%, 0.27%, 0.28%, 0.29%, and 0.30% w/v,

The LABA can be present as a salt or an ion-pair complex. Without being bound by theory, it is believed a LABA ion-pair complex can have increased residence time in the eye as compared to a LABA not administered as an ion-pair complex.

The methods herein can further include administering one or more muscarinic antagonist to an eye of the subject (e.g., affected eye, or non-affected eye, depending on the vision disorder). The formulations herein can further include one or more muscarinic antagonists. LAMAs include tiotropium, umeclidinium, and glycopyrollate. Non-limiting example of additional muscarinic antagonists include atropine, scopolamine, hydroxyzine, ipratropium, tropicamide, pirenzepine, diphenhydramine, doxylamine, dimenhydrinate, dicyclomine, flavoxate, oxybutynin, tiotropium, cyclopentolate, atropine methonitrate, trihexyphenidyl, tolterodine, solifenacin, darifenacin, benztropine, mebeverine, procyclidine, aclidinium and bromide salts thereof, other counter ion salts thereof, and combinations thereof. Optionally, the muscarinic antagonist is atropine. Optionally, the muscarinic antagonist is tiotropium. Optionally, the muscarinic antagonist is umeclidinium. Optionally, the muscarinic antagonist is aclidinium. Optionally, the muscarinic antagonist is glycopyrrolate. The muscarinic antagonist can be present in a formulation in a concentration of from about 0.0001% to about 10%.

Vision disorders in subjects that can be treated using the provided methods and formulations include subjects previously treated with atropine. For example, a subject previously treated with atropine can be selected and treated with a LABA. Administration of a LABA may lead to reduction in the need for atropine administration. For example, a dose of atropine used in a subject in combination with a LABA can be reduced relative to the dose of atropine administered to the subject prior to treatment with the LABA. The need for or use of atropine can be reduced or removed altogether, such that a subject previously treated with atropine prior to starting administration of a LABA no longer receives atropine while being treated with a LABA. The subject can experience a reduction of side effects associated with atropine use, such as mydriasis, dry eye, and cardiac side effects.

The methods can further include administering one or more α-adrenergic agonists to an eye of the subject. An α-adrenergic agonist may be combined with a LABA, a LAMA, a combination thereof, or a single drug that exhibits both activities. The formulations of herein can further include one or more α-adrenergic agonists. The α-adrenergic agonist can be selected from an α-1 agonist (e.g., methoxamine, midodrine, oxymetazoline, metaraminol, phenylephrine), an α-2 agonist (e.g., clonidine, guanfacine, guanabenz, guanoxabenz, guanethidine, xylazine, mizanidine, medetomidine, methyldopa, methylnorepinephrine, fadolmidine, dexmedetomidine), or other α-adrenergic agonists, such as amidephrine, amitraz, anisodamine, apraclonidine, brimonidine, cirazoline, detomidine, epinephrine, ergotamine, etilefrine, indanidine, lofexidine, medetomidine, mephentermine, metaraminol, methoxamine, mivazerol, naphazoline, norepinephrine, norfenefrine, octopamine, oxymetazoline, phenylpropanolamine, propylhexedrine, rilmenidine, romifidine, synephrine, talipexole, salts thereof, and combinations thereof. The α-adrenergic agonist is brimonodine. The α-adrenergic agonist can be present in a formulation in a concentration of from about 0.001% to about 10%.

The methods can also, optionally, further include administering one or more phosphodiesterase (PDE) inhibitors to an eye of the subject. The formulations herein can further include one or more phosphodiesterase (PDE) inhibitors. The phosphodiesterase inhibitor can be selected from non-selective or subtype-selective inhibitors. Exemplary non-limiting non-selective inhibitors can include, for example, theophylline, caffeine, aminophylline, IBMX (3-isobutyl-1-methylxanthine), paraxanthine, pentoxifylline, and theobromine. Exemplary non-limiting selective inhibitors can include, for example. PDE1-elective inhibitors, such as vinpocitine and the like; PDE2-selective inhibitors, such as EHNA (erythro-9-(2-hydroxy-3-nonyl)adenine), BAY 60-7550 (2-[(3,4-dimethoxyphenyl)methyl]-7-[(2R,3R)-2-hydroxy-6-phenylhexan-3-yl]-5-methyl-1H-imidazo[5,1-f][1,2,4]triazin-4-one), Oxindole, PDP (9-(6-Phenyl-2-oxohex-3-yl)-2-(3,4-dimethoxybenzyl)-purin-6-one), and the like; PDE3-selective inhibitors, such as inamrinone, milrinone, enoximone, anagrelide, cilostazol, pimobendan, and the like; PDE4-selective inhibitors, such as mesembrenone, rolipram, ibudilast, piclamilast, luteolin, drotaverine, roflumilast, apremilast, crisaborole, and the like; PDE5-selective inhibitors, such as sildenafil, tadalafil, vardenafil, udenafil, avanafil, dipyridamole, and the like; PDE7-selective inhibitors, such as quinazoline and the like; PDE10-selective inhibitors, such as papaverine and the like; and combinations thereof.

The methods can further include administering one or more α-adrenergic antagonists to an eye of the subject. An α-adrenergic antagonist may be added to a LABA, a LAMA, a combination comprising a β-adrenoceptor agonist and a LAMA, or a single drug that exhibits both activities. The formulations of herein can further include one or more α-adrenergic antagonists. The α-adrenergic agonist can be selected from the following α-1 antagonist non-limiting examples: phenoxybenzamine, phentolamine, prazosin, doxazosin, bunazosin, alfuzosin, terazosin, tamsulosin, yohimbine, labetalol, carvedilol, tolazoline, trazodone, mirtazapine, indoramin, urapidil, and idazoxan.

The route of administration of the herein provided formulations include administration to the periorbital skin that surrounds the anterior part of the globe. This provides a convenient and tolerable route of administration. Moreover, the periorbital skin provides a large drug reservoir, which facilitates steady drug delivery to the active site(s). The active ingredient can be a LABA, a LAMA, a single drug that exhibits both D-adrenoceptor agonist and muscarinic activities, an α-adrenergic agonist, a phosphodiesterase inhibitor, or permutations and combinations thereof.

The formulations can be in the form of a topical ocular formulation. Optionally, the topical ocular formulation can be in the form of an eye drop. Optionally, the topical ocular formulation can be in the form of a topical cream, gel, hydrogel, organogel, xerogel, lotion, nanocomposite hydrogel, foam, or a solution in an organic solvent, for topical application to the periorbital skin. Optionally, the formulations can be in the form of an implant, e.g., anterior chamber implants, subconjunctival implants, suprachoroidal implants; or an injectable formulation, e.g., for subconjunctival injection.

Formulations containing the compounds described herein can contain a physiologically compatible vehicle used in formulations for treating vision disorders. For example, the formulation can be an eye drop or an injectable. The vehicles can be selected from the known ophthalmic vehicles including, but not limited to, saline solution, water polyethers such as polyethylene glycol, polyvinyls such as polyvinyl alcohol and povidone, cellulose derivatives such as methylcellulose and hydroxypropyl methylcellulose, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate and salts such as sodium chloride and potassium chloride. The formulations can be in the form of a solution, a suspension, an ointment, gel or foam.

A subject can experience improvement or disappearance of one or more symptoms associated with a vision disorder, in an affected eye, or a collateral symptom in a non-affected eye (such as eye strain) when the herein provided methods are employed. For example, non-limiting symptoms that can improve include increased regularity in eyeball shape (e.g., less elongation), improved visual acuity (e.g., achieving a visual acuity of better than 20/20, better than 20/25, better than 20/30, better than 20/40, better than 20/50, better than 20/70, better than 20/100, or better than 20/200). A method is provided for improving the visual acuity in an affected eye of a subject having myopia, comprising administering a LABA, a LAMA, a combination comprising a LABA and a LAMA or a single hybrid drug that exhibits both β-adrenoceptor agonist and muscarinic antagonist properties to an eye of the subject. A LABA, a LAMA, a combination comprising a LABA and a LAMA, or a single drug that exhibits both activities can provide consistent reduced resting tone of the ciliary muscle, thereby reducing the ciliary muscle contractile and spasmodic episodes in an affected eye of a subject having pre-myopia or pseudo-myopia. Optionally, the opposing effect of a LABA, a LAMA, a combination comprising a LABA and a LAMA, or a single drug that exhibits both activities on ciliary muscle contraction would also similarly treat myopia or cycloplegia. Optionally, a subject's need for using spectacles or contact lenses can be reduced and/or eliminated. Optionally, a subject's prescription strength of corrective lenses can be reduced. Optionally, use of a LABA, a LAMA, a combination comprising a LABA and a LAMA, or a single drug that exhibits both activities can slow the progression of the need for increased prescription strength of corrective lenses in a subject with the passage of time. Optionally, the risk of retinal detachment, myopia-induced retinopathies, and glaucoma would be decreased or removed. Optionally, use of a LABA, a LAMA, a combination comprising a LABA and a LAMA, or a single drug that exhibits both activities can slow, stop, or prevent the increase in axial length of the globe that may occur in a subject having myopia or amblyopia.

A number of embodiments of the invention have been described. Nevertheless, it will be understood that various modifications may be made without departing from the spirit and scope of the invention. For example, different amounts of a LABA, a LAMA, a combination comprising a LABA and a LAMA, or a single drug that exhibits both activities and forms thereof may be used in the methods described herein. Accordingly, other embodiments are within the scope of the following claims.

### EXAMPLES

### Example 1. Isolated Ciliary Muscle from Non-Human Primates

A total of 4 longitudinal strips of ciliary muscle were obtained from one monkey eye. Four tissue baths were used concurrently for each experiment. The monkey ciliary muscle preparations were incubated in Krebs bicarbonate buffer, aerated with 95% O₂/5% CO₂, containing 10⁻⁶ M indomethacin. The preparations were placed under 200 mg tension with adjustment and allowed to equilibrate for 30 minutes. The temperature was maintained at 37° C. throughout the experiment. After equilibration, the four tissue baths received (50 μL×10⁻³M) 10⁻⁶ M carbachol (sufficient to produce a mid-level 40-60% maximal response). If 10⁻⁶ M carbachol was insufficient to cause a meaningful contraction, then (50 μL×3×10⁻³M) 3×10⁻⁶ M or (50 μL×10⁻²M) 10⁻⁵M was used. The contractile response was allowed to become stable. Tissue tension was recorded on a multi-channel electrophysiological recorder.

The drugs of interest, salmeterol (JV-M1) and tiotropium (JV-M5), were added cumulatively at ascending graded concentrations in 50 μL aliquots as follows: a 50 μL aliquot of 10⁻⁵ M (->10⁻⁸ M); a 50 μL aliquot of 10⁻⁴ M (->10⁻⁷ M); a 50 μL aliquot of 10⁻³M (->10⁻⁶ M); a 50 μL aliquot of 10⁻²M (->10⁻⁵M).

In one experiment, salmeterol and tiotropium were added in combination.
When cumulative addition of the drug solutions was completed, the drugs were washed-out by flushing through with buffer three times at a selected volume.
Four tissue baths again received 10⁻⁶ M (or selected concentration) carbachol and a stable contractile response was established.

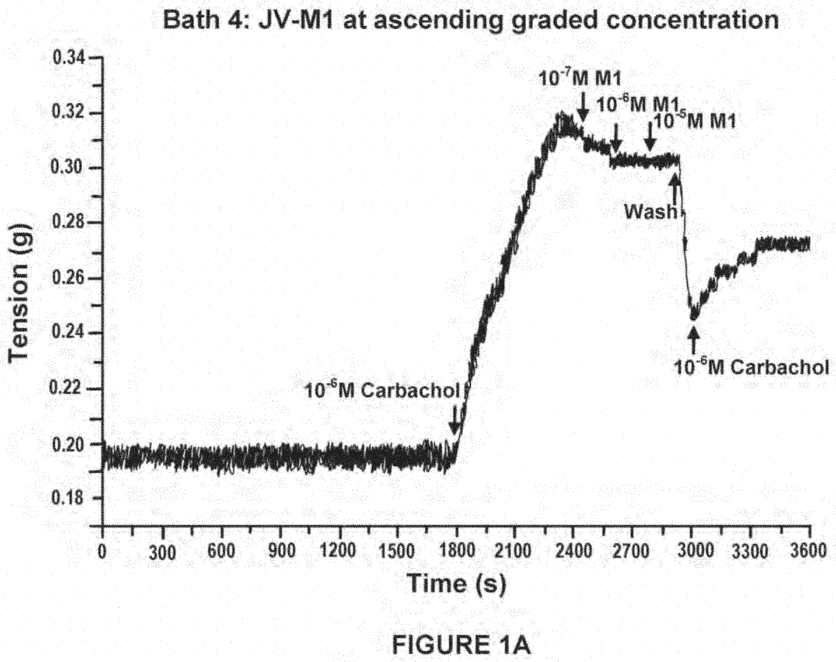

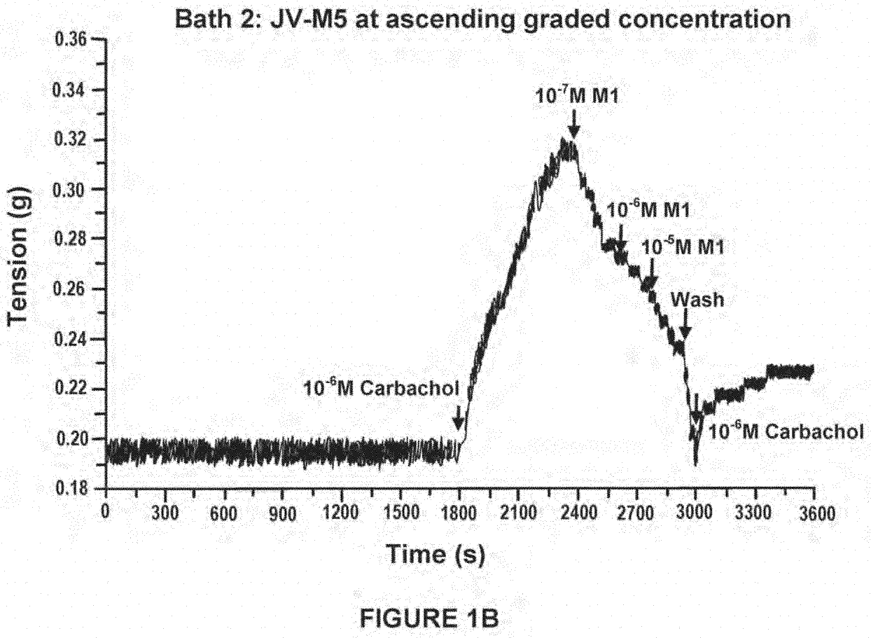

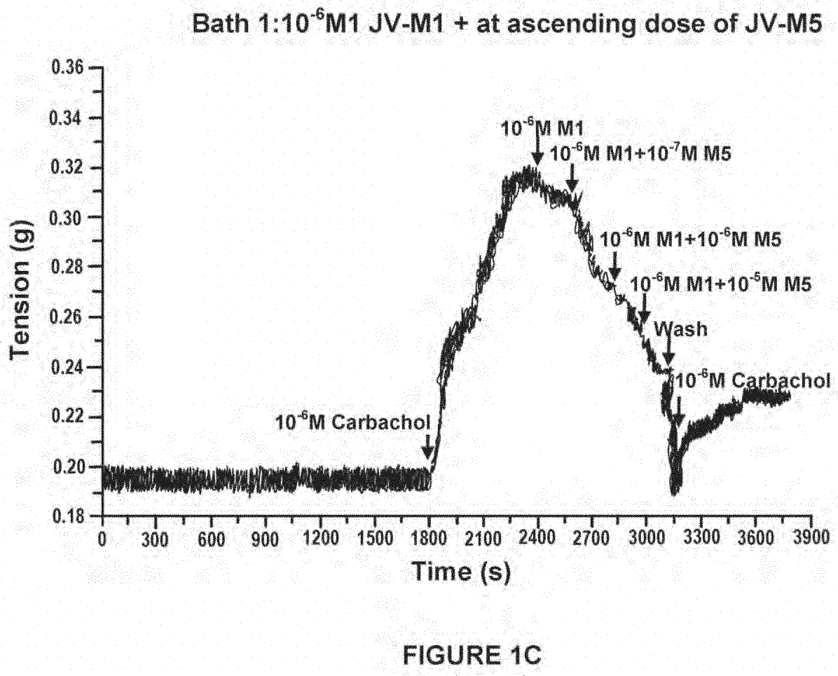

The effects of salmeterol, tiotropium, and a combination thereof on the pre-contracted monkey ciliary muscle preparation are shown in FIGS. 1A, 1B and 1C, respectively.

The β₂-adrenoreceptor agonist salmeterol and the muscarinic antagonist tiotropium produced a similar dose-related decrease ciliary muscle tone (FIG. 1A and FIG. 1B), although the magnitude of relaxation for tiotropium was greater than that recorded for salmeterol. Following wash-out, the contractile effects of a further application of carbachol were blunted by both salmeterol and tiotropium, which is consistent with their long acting properties (FIG. 1A and FIG. 1B). It also suggests that salmeterol is more effective pre-dosed than post-dosed. A combination of salmeterol and tiotropium was also highly effective in reducing ciliary smooth muscle tone (FIG. 1C).

Vision disorders, where accommodation is aberrant due to ciliary muscle dysfunction, require subtle treatment. In myopia involving excessive ciliary smooth muscle tone, treatment should rely on modest relaxation of the ciliary muscle such that the eye may still accommodate on close work. Pronounced ciliary smooth muscle relaxation would produce hyperopia and alternative and equally problematic eyesight disorders. The objective of optimal treatment for myopia would be satisfied by salmeterol (and other LABAs) and low doses of tiotropium (and other LAMAs and muscarinic antagonists), and combinations thereof.

### Example 2. Ocular Bioavailability Salmeterol and Tiotropium Following Application to the Periorbital Skin of Cynomolgus Monkeys

Tiotropium and salmeterol were prepared in phosphate buffered saline, salmeterol as a suspension. A total median amount of 208 μg tiotropium and 155 μg salmeterol were achieved by wiping the solutions by micro-brush in multiple discrete applications to the upper and lower periorbital skin of one eye. The animals were euthanized at predetermined time points post-dosing: 1, 2, 4, 24 hours. A blood sample and ocular tissue specimens were then taken and prepared for LC/MS/MS analysis. The results for tiotropium and salmeterol are shown in Tables 1 and 2, respectively.

TABLE 1
Quantification of tiotropium levels (M concentrations) in the
ciliary muscle and plasma at predetermined times post-dosing.

| | Time (hr) post-dosing | | | |
|---|---|---|---|---|
| | 1 | 2 | 4 | 24 |
| Plasma | 4.5 × 10⁻⁹M | BLQ | BLQ | BLQ |
| Ciliary Muscle | 4.9 × 10⁻⁸M | 7.7 × 10⁻⁸M | 5.4 × 10⁻⁹M | 0 |

(BLQ = below limit of quantitation)

TABLE 2
Quantification of salmeterol levels (M concentrations) in the
ciliary muscle and plasma at predetermined times post-dosing.

| | Time (hr) post-dosing | | | |
|---|---|---|---|---|
| | 1 | 2 | 4 | 5 |
| Plasma | BLQ | BLQ | BLQ | BLQ |
| Ciliary Muscle | 1.7 × 10⁻⁸M | 8.4 × 10⁻⁹M | 3.3 × 10⁻⁸M | 5.8 × 10⁻⁸M |

(BLQ = below quantifiable limit)

Application to the periorbital skin of tiotropium (Table 1) and salmeterol (Table 2), employing non-optimized aqueous formulations, allowed both drugs to access the ciliary muscle in pharmacologically active concentrations. This would allow an appropriate modulation of ciliary muscle tone to suitably correct vision disorders. It should be noted that adequate drug levels were achieved in the ciliary muscle with minimal or absent systemic exposure for tiotropium and salmeterol, respectively.

1. A method of treating a vision disorder in a subject comprising administering a long-acting β-adrenoceptor agonist to one or both eyes of the subject to treat the vision disorder in the subject.

2. The method of claim 1, wherein the administration is ocular or periocular administration.

3. The method of claim 1, wherein the vision disorder is a disorder that can be treated by ciliary muscle modulation in an eye of the subject.

4. The method of claim 1, wherein the disorder is selected from the group consisting of myopia, pre-myopia, pseudomyopia, antimetropia, exophoria, amblyopia, anisometropia, esoptropia, exoptropia, Duane's syndrome I, Duane's syndrome II, Brown's syndrome, ocular complications from surgery, injury to the eye or fracture of the orbital bone, a vision disorder resulting from retinal detachment, a vision disorder resulting from cataract, a vision disorder associated with diabetes, a vision disorder associated with myasthenia gravis, and a vision disorder associated with Grave's disease.

5. The method of claim 1, wherein the long-acting β-adrenoceptor agonist is administered to a non-affected eye of the subject.

6. The method of claim 5, wherein the disorder is selected from the group consisting of anisometropia, amblyopia, esoptropia, and exoptropia.

7. The method of claim 1, wherein the long-acting β-adrenoceptor agonist is administered to an affected eye of the subject.

8. The method of claim 7, wherein the disorder is selected from the group consisting of myopia, pseudomyopia, antimetropia, and exophoria.

9. The method of claim 1, wherein the long-acting β-adrenoceptor agonist is administered to the eye as a topical ocular formulation.

10. The method of claim 1, wherein the long-acting β-adrenoceptor agonist is administered to the eye as an eye drop.

11. The method of claim 1, wherein the long-acting β-adrenoceptor agonist is administered to the eye by topical application to the periorbital skin.

12. The method of claim 1, wherein the long-acting β-adrenoceptor agonist is administered to the eye as an implant.

13. The method of claim 12, wherein the implant is an ocular interior chamber implant or a suprachoroidal implant.

14. The method of claim 1, wherein the long-acting β-adrenoceptor agonist is selected from the group consisting of albuterol, formoterol, salmeterol, vilanterol, indacaterol, arformeterol, olodaterol, and combinations thereof.

15. The method of claim 1, wherein the long-acting β-adrenoceptor agonist is batefenterol.

16. The method of claim 1, further comprising administering a muscarinic antagonist to an eye of the subject.

17. The method of claim 16, wherein the muscarinic antagonist is administered to a same eye of the subject to which the long-acting β-adrenoceptor agonist is administered.

18. The method of claim 16, wherein the muscarinic antagonist is selected from the group consisting of atropine, scopolamine, hydroxyzine, ipratropium, tropicamide, pirenzepine, diphenhydramine, doxylamine, dimenhydrinate, dicyclomine, flavoxate, oxybutynin, cyclopentolate, atropine methonitrate, trihexyphenidyl, tolterodine, solifenacin, darifenacin, benztropine, mebeverine, procyclidine, and combinations thereof.

19. The method of claim 17, wherein the long-acting muscarinic antagonist selected form the group consisting of tiotropium, umeclidinium, aclidinium and glycopyrronium and salts thereof.

20. The method of claim 18, wherein the muscarinic antagonist is atropine.

21. The method of claim 16, wherein the subject was previously treated with atropine.

22. The method of claim 20, wherein a dose of atropine used in combination with the long-acting β-adrenoceptor agonist is reduced relative to the dose of atropine administered to the subject prior to treatment with the long-acting β-adrenoceptor agonist.

23. The method of claim 1, further comprising administering an α-adrenergic agonist to an eye of the subject.

24. The method of claim 23, wherein the α-adrenergic agonist is selected from the group consisting of methoxamine, midodrine, oxymetazoline, metaraminol, phenylephrine, clonidine, guanfacine, guanabenz, guanoxabenz, guanethidine, xylazine, mizanidine, medetomidine, methyldopa, methylnorepinephrine, fadolmidine, dexmedetomidine, amidephrine, amitraz, anisodamine, apraclonidine, brimonidine, cirazoline, detomidine, epinephrine, ergotamine, etilefrine, indanidine, lofexidine, medetomidine, mephentermine, metaraminol, methoxamine, mivazerol, naphazoline, norepinephrine, norfenefrine, octopamine, oxymetazoline, phenylpropanolamine, propylhexedrine, rilmenidine, romifidine, synephrine, talipexole, salts thereof, and combinations thereof.

25. The method of claim 24, wherein the α-adrenergic agonist is brimonodine.

26. The method of claim 1, further comprising administering a phosphodiesterase (PDE) inhibitor to an eye of the subject.

27. The method of claim 26, wherein the phosphodiesterase (PDE) inhibitor is selected from the group consisting of vinpocitine, erythro-9-(2-hydroxy-3-nonyl)adenine (EHNA), 2-[(3,4-dimethoxyphenyl)methyl]-7-[(2R,3R)-2-hydroxy-6-phenylhexan-3-yl]-5-methyl-1H-imidazo[5,1-f][1,2,4]triazin-4-one, oxindole, 9-(6-Phenyl-2-oxohex-3-yl)-2-(3,4-dimethoxybenzyl)-purin-6-one (PDP), inamrinone, milrinone, enoximone, anagrelide, cilostazol, and pimobendan, mesembrenone, rolipram, ibudilast, piclamilast, luteolin, drotaverine, roflumilast, apremilast, crisaborole, sildenafil, tadalafil, vardenafil, udenafil, avanafil, dipyridamole, quinazoline, papaverine, and combinations thereof.

28. The method of claim 26, wherein the phosphodiesterase inhibitor is theophylline.

29. An ocular formulation comprising a long-acting β-adrenoceptor agonist.

30. The formulation of claim 29, wherein the β-adrenoceptor agonist is selected from the group consisting of albuterol, formoterol, salmeterol, vilanterol, indacaterol, arformeterol, olodaterol, and combinations thereof.

31. The formulation of claim 29, wherein the long-acting β-adrenoceptor agonist is present at a concentration of from about 0.001% to about 10% w/v.

32. The formulation of claim 29, wherein the formulation is a topical ocular formulation.

33. The formulation of claim 32, wherein the topical ocular formulation is in the form of an eye drop.

34. The formulation of claim 32, wherein the topical ocular formulation is in the form of a periorbital formulation.

35. The formulation of claim 29, wherein the formulation further comprises a muscarinic antagonist.

36. The formulation of claim 35, wherein the muscarinic antagonist is a long-acting muscarinic antagonist is selected from the group consisting tiotropium, aclidinium, umeclidinium, glycopyrroium and salts thereof.

37. The formulation of claim 35, wherein the muscarinic antagonist is selected from the group consisting of atropine, scopolamine, hydroxyzine, ipratropium, tropicamide, pirenzepine, diphenhydramine, doxylamine, dimenhydrinate, dicyclomine, flavoxate, oxybutynin, cyclopentolate, atropine methonitrate, trihexyphenidyl, tolterodine, solifenacin, darifenacin, benztropine, mebeverine, procyclidine, bromide, and combinations thereof.

38. The formulation of claim 37, wherein the muscarinic antagonist is atropine.

39. The formulation of claim 35, wherein the muscarinic antagonist is present at a concentration of from about 0.001% to about 10% (w/v).

40. The formulation of claim 35, wherein the topical ocular formulation is in the form of an eye drop.

41. The formulation of claim 35, wherein the topical ocular formulation is in the form of a periorbital formulation.

42. A method for improving visual acuity in an affected eye of a subject having myopia, comprising administering a long-acting β-adrenoceptor agonist, a muscarinic antagonist, or a combination thereof to the affected eye of the subject to improve visual acuity in the affected eye of the subject.
